(19) United States
(12) Patent Application Publication
(10) Pub. No.: US 2005/0042236 A1
(43) Pub. Date: Feb. 24, 2005

(54) Plant extract active as an immunostimulating agent

(76) Inventors: Ravshan Inoyatovich Ataullakhonov, Moscow (RU); Tatiana Mikhailovna Melnikova, Moscow (RU); Rakhim Musaevich Khaitov, Moscow (RU); Alexei Vasilevich Pichugin, Moscow (RU)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/495,730

(22) PCT Filed: Nov. 15, 2002
(86) PCT No.: PCT/GB02/05142

(30) Foreign Application Priority Data
Nov. 16, 2001 (RU) 2001130780
May 22, 2002 (GB) 0211806.5

(51) Int. Cl. A61K 45/00; A61K 47/00

(52) U.S. Cl. 424/278.1

(57) ABSTRACT

A substance having immunostimulating, anti-viral and/or anti-bacterial activities, obtainable by extraction from mashed plant raw material of the Dioscoreaceae, Plantaginaceae or Solanaceae family with water, and which is a water-soluble acidic peptidoglycan having a molecular weight of 1200-40000 kD, and a glucose to uronic acid weight ratio of 1 to 2-4.

### FIELD OF THE INVENTION

The invention relates to a plant extract active as an immunostimulating agent.

### BACKGROUND OF THE INVENTION

A biologically active polysaccharide has been produced by mashing sprouts of potato bulbs, extraction with boiling water, maintenance at 20° C. for 16-18 hours, separation of the extract, keeping for no less than 20 days, fractionation according to molecular weight, and extraction of a substance having a molecular weight of more than 10000 Dalton, followed by concentration and drying (RU 2108800 C1, A 61 K 35178, 20.04.1998). The substance produced in this way possesses anti-viral and anti-bacterial activity.

Polysaccharides that possess immunostimulating activity have been produced by treating plant material with aqueous formaldehyde, keeping in aqueous acid, extraction of pectin polysaccharides with aqueous ammonium oxalate, and lyophilisation. Fresh water flower plants are used as raw material, for example any species of weed such as Lemna spp, and finely cut ‘above-the surface’ parts of higher plants, for example Oberna behen (L) (RU 2149642 C1, A 61K 35/78, 27.05.2000).

L. A. Chekanovskaya and A. V. Generalov disclose the extraction and characteristics of a biologically active preparation ‘Gamma-plant’ from sprouts of potato Solanum tuberosum; see Chem. Pharm Journal (2000) 34(3): 51-56. Its production involves mashing of the raw material, extraction with boiling water, centrifugation of the aqueous extract, concentration and precipitation with acetone, refining and drying of the whole product. Refining is conventional, and comprises dialysis, gel-filtration on AcA44, ion-exchange chromatography, and HPLC at the final stage. The substance produced using this method is a glycoprotein having a molecular weight of 70 kD, comprising 90% carbohydrate and 10% protein. The carbohydrate portion of gamma-plant consists of 70% glucose, 3.7% arabinose, 2.08% xylose, 6.84% galactose, 0.58% mannose, 1% aminosaccharides and 5.1% uronic acids.

### SUMMARY OF THE INVENTION

According to the present invention, a novel substance having immunostimulating, anti-viral and anti-bacterial activities, can be extracted from mashed raw plant material of the Dioscoreaceae, Plantaginaceae or Solanaceae families. In particular, an aqueous extract is centrifuged, then concentrated and precipitated with 96% ethanol in the presence of sodium chloride. The precipitate is dissolved and reprecipitated using saline or acid and the raw acidic peptidoglycan thus obtained is treated with alkali or a saturated solution of an alkali metal salt; the purified acidic peptidoglycan is refined using gel-chromatography, and is then dried.

Such a method, depending on a particular order of operations, gives a new substance having strong immunostimulating, anti-viral and anti-bacterial activities, and which is a water-soluble acidic peptidoglycan having molecular weight of 1200-40000 kD, and glucose to uronic acid weight ratio of 1 to 2-4. A pharmaceutical composition may comprise the peptidoglycan together with one or more pharmaceutically acceptable fillers and/or carriers.

### DESCRIPTION OF PREFERRED EMBODIMENTS

The given method preferably uses leaves, stems, roots, bulbs and/or sprouts of plants. Different parts of the plant may be used, at any stage of its development to maturity.

During secondary sedimentation, cetavion or calcium chloride is preferably used as a salt. Organic or inorganic acids, or acidic non-organic salts, may be used as acidic agents. The secondary sedimentation allows raw acidic peptidoglycan to be separated from various accompanying polysaccharides and proteins.

The acidic peptidoglycan product may be purified using, for example, gel-chromatography; TSK HW-75 F, Sepharose 2B or 4B CL are suitable chromatography agents.

The peptide portion of the novel substance may constitute 13±3% of the mass of the whole peptidoglycan molecule. The amount of peptide was measured using Lowry method using bovine serum albumin as a standard (Lowry et al, J. Biol. Chem (1951) 193:265-275). The analysis shown in Table 1 was obtained (showing 5 predominant amino-acids).

TABLE 1

| Amino-acid | % mass |
|---|---|
| Asp | 10.6 |
| Thr | 5.6 |
| Ser | 10.3 |
| Glu | 11.1 |
| Gly | 8.9 |
| Ala | 12.5 |
| Val | 5.9 |
| Ile | 2.8 |
| Leu | 5.0 |
| Tyr | 1.3 |
| Phe | 2.6 |
| His | 19.0 |
| Lys | 1.8 |
| Arg | 2.6 |

The polysaccharide part of the molecule was found to consist of the following:

| | |
|---|---|
| galacturonic acid | 18 ± 6% |
| glucose | 9 ± 3% |
| galactose | 5.5 ± 2% |
| mannose | 0.7 ± 0.25% |
| arabinose | 3.8 ± 1.3% |
| rhamnose | 1.9 ± 0.9% |

The acidic sugars were determined on the basis of colour reaction with 3,5-dimethylphenol in concentrated sulphuric acid (Usov et al, Botanica Marina (1995), 38:43-51). The predominant presence of galacturonic acid in the acidic peptidoglycan was found using gas-liquid chromatography (GLC) of uronic acids in the form of 3-methylsilyl derivatives (Albersheim, Methods Enzymol. (1987) 118: 3-40). The neutral sugars were detected using GLC analysis of saccharides in their polyol acetate forms (Albersheim, supra).

As will be evident from the results reported below, a substance of the invention has various beneficial properties. It can be used in therapy (including propylaxis) of a condition, in humans or other mammals or animals, that can be ameliorated by any of enhanced antibody synthesis or activated macrophages, monocytes, NK-cells, granulocytes or cytokine or interferon synthesis. Such conditions are known to those skilled in the art.

In use, the substance will typically be formulated as a pharmaceutical composition comprising an acceptable carrier. Examples of suitable carriers (which may be liquid) are well known in the art, and will be chosen according to the desired route of administration. The route and also the dosage can readily be chosen by one skilled in the art, depending on the usual factors such as the severity of the condition, the state of the patient, other therapies etc. Guidance on these factors is provided by the Examples, below.

The following Examples illustrate this invention.

### EXAMPLE 1

Plant cells of the root part of dioscorea from the Dioscoreaceae family (D. cuacasia Lipsky) were grown in a culture medium. At the end of the incubation period, the cell mass was separated by filtering, and the supernatant was concentrated, dialysed against distilled water and freeze-dried.

500 ml of distilled water was added to 5 g of dry mixture produced from culture liquid. Extraction was performed at room temperature for 3 hours. Non-soluble sediment was discarded using centrifugation. Supernatant was concentrated to 200 ml in volume; 200 mg of sodium chloride was dissolved in it, and then 600 ml of 96% ethanol was added. The sediment was extracted by centrifugation at 4000 rpm for 30 minutes. The sediment was then washed using 50 ml of 96% ethanol, centrifuged and dried under an air flow.

1 g of dry product, obtained as described above, was diluted in 200 ml of distilled water by mixing at room temperature for 1 hour. To this solution, 5 ml of 10% solution of trichloracetic acid was added. The resultant precipitate, an acidic raw peptidoglycan, was separated by centrifugation, and washed with water. Then 50 ml of distilled water was added and, whilst mixing, 25% of ammonia solution was added dropwise, until complete solubilisation of the sediment was achieved.

The produced solution was introduced into a TSK HW-75F column for gel-chromatography. Fractions were eluted from the column using water. The first high molecular peak, having a molecular weight between 1200 and 40000 kD, was collected. The fraction was concentrated on a rotary evaporator and lyophilised.

The yield of acidic peptidoglycan was 39 mg. The weight ratio of glucose to uronic acids was 1:3. The peptide content was 11.2%.

### EXAMPLE 2

100 g of fresh stems of dioscorea from the Dioscoreaceae family (D. cuacasia Lipsky) was mashed. 500 ml distilled water was added, and the mashed product extracted at 30-40° C. for 5 hours. The mixture was squeezed using a mechanical press, and the aqueous extract was collected, dialysed and concentrated to 100 ml in volume using an evaporator. 100 mg sodium chloride was added to the concentrate, after which 300 ml of 96% ethanol was also added. The precipitate was extracted using centrifugation at 4000 rpm for 30 minutes. The sediment was then washed using 30 ml of 96% ethanol, centrifuged and dried under an air flow.

1 g of dry sediment obtained as above was diluted in 200 ml of distilled water by mixing at room temperature for 1 hour. The insoluble part was discarded and 5 ml of concentrated HCl was added to the supernatant. The resultant sediment was separated by centrifugation and washed in water. 50 ml distilled water was then added and, whilst mixing, 25% of ammonia solution was added dropwise, until complete solubilisation of the sediment was achieved.

Fractions were eluted from the column using water. The first high molecular peak, having a molecular mass between 1200 and 40000 kD, was collected. The fraction was concentrated on an evaporator and then freeze-dried.

The yield of acidic peptidoglycan was 16 mg. The weight ratio of glucose to uronic acids was 1:2.2. The peptide content was 14.7%.

### EXAMPLE 3

100 g of freshly harvested plantain leaves, from the Plantaginaceae family (P. major L.), were mashed in 500 ml of distilled water and extracted whilst heating at 30-40° C. for 4 hours. The mixture was squeezed using a mechanical press, and the aqueous extract harvested, dialysed and concentrated to 100 ml using an evaporator. 100 mg sodium chloride was added to the concentrate, after which 300 ml of 96% ethanol was also added. The precipitate was extracted using centrifugation at 4000 rpm for 30 minutes. The sediment was then washed using 30 ml of 96% ethanol, centrifuged and dried under an air flow.

1 g of dry product was diluted in 200 ml of distilled water by mixing at room temperature for 1 hour. The insoluble part was discarded and 5 ml of concentrated HCl was added to the supernatant. The produced sediment was separated by centrifugation and washed in water. Then 50 ml of distilled water was added and, whilst mixing, 25% of ammonia solution was. added dropwise, until complete solubilisation of the sediment was achieved.

The resultant solution was introduced into a TSK HW-75F column, for gel-chromatography. Fractions were eluted from the column using water. The first high molecular peak, having a molecular mass between 1200 and 40000 KD, was collected. The fraction was concentrated on an evaporator and then freeze-dried.

The yield of acidic peptidoglycan was 16 mg. The weight ratio of glucose to uronic acids was 1:2.7. The peptide content was 13.8%.

### EXAMPLE 4

Example 3 was repeated except that, instead of using 5 ml concentrated HCl for extracting acidic peptidoglycan, 200 ml of a concentrated solution of NH₄NO₃ or (NH₄)₂SO₄ was used.

The yield of the final product was 14 mg. The mass correlation between glucose and uronic acids was 1:2.4. The peptide content was 15.8%.

### EXAMPLE 5

5 kg of potato sprouts.from the Solanaceae family (S. tuberosum) were mashed with 10 L water and extracted at room temperature, whilst being stirred, for 2 hours. The mixture was squeezed using a mechanical press. The water extract was dialyzed and concentrated to 1 litre by ultra-filtration using a 10 kD hollow-fibre cartridge.

1 g sodium chloride was added to the concentrate, after which 300 ml of 96% ethanol was also added. The precipitate was extracted by centrifugation at 4000 rpm for 30 minutes. The sediment was then washed using 30 ml of 96% ethanol, centrifuged and dried under an air flow.

10 g of dry product was diluted in 1 litre of distilled water whilst stirring at room temperature for 1 hour. The insoluble part was discarded, and 150 ml of 5% calcium chloride was added to the supernatant. The sediment, an acidic raw peptidoglycan, was separated by centrifugation. It was then dissolved in a saturated solution of sodium chloride, with stirring at 50° C., until the polymer was fully solubilised.

The resultant solution was introduced into a TSK HW-75F column, for gel-chromatography. Fractions were eluted from the column using water. The first high molecular peak, having a molecular weight between 1200 and 40000 kD, was collected. The fraction was concentrated on an evaporator and then freeze-dried.

The yield of acidic peptidoglycan was 170 mg. The weight ratio of glucose to uronic acids was 1:4.

### EXAMPLE 6

Example 4 was repeated, except that potato tubers were used, and 9% cetavlon solution was used as a salt in the secondary sedimentation. The output of the acidic peptidoglycan was 114 mg. The weight ratio of glucose and uronic acids was 1:3.5. The peptide content was 16%.

### EXAMPLE 7

21.4 mg of acidic peptidoglycan obtained as in Example 3 was subjected to partial hydrolysis in 0.1 M trifluoroacetic acid at 100° C. over 2 hours. The sediment formed during hydrolysis was obtained by centrifugation (13000 rpm, 2 minutes), washed using trifluoroacetic acid and water, and then lyophylised. The yield of sediment (encoded L-1) was 1.5 mg. Soluble material left after removal of the sediment was dried by vacuum vaporization, and then solubilised in ethanol and dried to remove traces of trifluoroacetic acid. The remainder was dissolved in 3 ml water, and the insoluble material was precipitated by centrifugation, washed with water and lyophilised.

The yield of sediment (L-2) was 1.5 mg. The total yield of sediments L-1 and L-2 was about 14% of the initial mass of acidic peptidoglycan. It was found that the sediments were represented by lipids.

Methanolysis of sediments was performed as follows. The samples of 0.8 mg L-1 and 0.9 mg L-2 were each mixed with 1 ml dry methanol, and then 0.1 ml acetyl chloride was added (cold) into each of above reaction mixtures. The reaction mixtures were heated in soldered glass ampoules at 100° C. for 4 hours, which led to complete solubilisation of the sediments. The reaction mixtures were dried by vacuum vaporization, then 1 ml of chloroform was added and the solutions were examined using gas-liquid chromatography (GLC) and a chromatomass-spectrometer.

The GLC-spectra of both samples (L-1 and L-2) were identical; each spectrum had 2 signals. GLC was performed on Carlo Fractovap Series 4200 (gas-carrier He, injector temperature 280° C., column—Ultra-1 (25 m×0.2 mm×0.33 μm).

Mass-spectra showed the presence of methyl esters of palmitic and stearic acids in a ratio 2:1. An ionic Capture ITD-700 (Finnigan MAT, electronic hit 70 EV, mass range m/z 39-450, scanning velocity 1 scan/sec, retention accumulation 240 sec) mass-spectrometer was used.

### EXAMPLE 8

Activation of Antibody Synthesis

This Example shows that injection of acidic peptidoglycan (APG) simultaneously with foreign antigen leads to a substantial intensification of antibody production, specific to the injected antigen. In the experiments, mice CBA, C57BI/6, (CBAxC57BI/6)F₁ and BALB/c were used. Bovine serum albumin (BSA), egg albumin (EA) or sheep erythrocytes (SE) was used as antigen for immunisation of mice.

Erythrocytes of defibrinated sheep blood were washed three times by centrifugation (1000 rpm, 10 m)in 50-fold volume of Hanks' solution, and were then re-suspended in the same solution. Mice were immunised intraperitoneally with 2 ml SE, 200 μg BSA or 50 μg EA. The latter two antigens were injected twice, with a 2-4 week interval between immunisations.

The appropriate amount of APG was dissolved in Hank's solution or in physiological saline, 2-3 hours prior to use. APG was administered to mice in doses of between 1 and 1000 μg. When. mice were immunised with BSA, EA or SE, APG was administered simultaneously with the antigen.

The intensity of the immune response in mice which were immunised with SE was determined according to the number of antibody-forming cells (AFC), detected in the spleen cell suspensions using the method of Jerne and Nordin (1963). AFC were detected 4-5 days following immunisation. Presence of antibodies specific to EA or BSA in blood serum of mice was detected using a solid-phase enzyme-immunoassay (EIA). Isotype of antibodies specific to BSA was determined by EIA using secondary rabbit antibodies specific to mouse IgM, IgG1, IgG2a, IgG2b or IgG3.

The level (EIA-titre) of antibodies specific to BSA in blood serum of mice following immunisation with BSA and APG reached 1:20 000, while the level of response to immunisation using BSA alone was much lower and reached an EIA titre 1:500 only. The longevity of the secondary immune reaction to BSA also increased if the antigen was combined with APG. In this case, antibodies of IgG1, IgG2a and IgG3 isotypes were dominating. Production. of IgM and IgG2b specific to BSA increased to a lesser extent under the influence of APG. The adjuvant effect largely depended on the APG dose. A dose of 10 μg APG was optimal for enhancing of primary immune response to SE, while a dose of 1 μg APG was optimal for stimulation of secondary immune response to BSA.

Influence of APG on production of EA-specific antibodies in BaBb/c mice is demonstrated by the data shown in Table 2. The lipopeptide was Pam₃Cys-Ser-Lys₄ a well-known immunoadjuvant.

TABLE 2

| | | | | Serum Titer |
|---|---|---|---|---|
| | | | | of EA- |
| | | | Route of | specific |
| Antigen | Preparation | Dose | Injection | Antibodies |
| EA, 50 μg | — | — | — | 13,000 |
| EA, 50 μg | APG | 20 μg | Intraperitoneal | 22,000 |
| EA, 50 μg | lipopeptide | 20 μg | Intraperitoneal | 20,000 |
| EA, 50 μg | Freund's adjuvant | 250 μl | Intraperitoneal | 33,000 |

### EXAMPLE 9

Activation of Tissue Macrophages

This Example shows that mouse tissue macrophages harvested from a peritoneal cavity exudates are strongly activated by in vitro cultivation in presence APG. The activation was seen in their morphology (cells changed their size and shape) as well as in their metabolic and enzymatic activities. (CBAxC57BI/6)F₁ mice were injected intraperitoneally using 3 ml of medium 199. Peritoneal exudate was collected within 5 minutes of injection. Exudates from 10-15 mice were harvested into the same siliconised centrifuge tube and spun down at 1000 rpm for 10 minutes. The cell pellet was re-suspended, adjusting the concentration to 2-2.5 mln peritoneal exudates cells (PEC) per ml.

Production of Oxidative Radicals by Macrophages

1 ml of cell suspension of PEC was poured into a chemiluminograph test tube and incubated for 2 hours at 37° C. in a humidified atmosphere of 5% CO₂ in air. After the incubation, non-adherent cells were washed away using medium 199. Then 1 ml of complete culture medium (RPMI-1640, 10% FCS, 2 mM L-glutamine, and 10 μg/ml gentamycin) containing APG in concentrations between 0 and 50 μg per ml, was poured into each tube on top of adherent cells attached to the tube. The tube was then incubated for 24 hours. After the incubation, culture medium was replaced by 0.5 ml of buffer solution (pH 7.2), prepared from Hank's solution (without phenol red), supplemented with 5 mM glucose, 10 mM HEPES-buffer and 0.62 mM luminol (Sigma Chemical Co).

Production of oxidising radicals by adherent macrophages was assessed as levels of spontaneous and zymozan-induced chemiluminescence. The results of the sum of three experiments are represented in an average number of impulses per minute per 1 mln cells. Incubation of adherent macrophages in the presence of APG over 24 hours did not influence the level of their spontaneous chemiluminescence, but increased considerably (up to 50%) the ability of cells to produce oxidising metabolites in response to zymozan, a component of microorganisms' cell wall. The increase of chemiluminescent activity of macrophages starts already at 0.2 μg/ml APG and reaches its maximum at 5.5 μg/ml APG. This dose-dependence correlates well with APG concentration, that leads to a maximum activation of macrophages assessed by their morphological changes.

Activity of 5′-nucleotidase (5′-NTDase) on the Membrane of Macrophages

Detection of the level of 5′-NTDase enzyme is one of the highly informative methods of assessment of macrophage activation. It is known that decrease in this enzyme activity under the influence of immunomodulators well correlates well with their immunoenhancing and anti-infective efficacies.

In order to determine the influence of APG on the level of 5′-NTDase of peritoneal macrophages, (CBAxC57BI/6)F₁, mice were intraperitoneally injected with 30 μg of APG in 0.5 ml of physiological saline. The control mice were injected with 0.5 ml of physiological saline without APG. 24 hours following the injection, the cells of peritoneal exudate were harvested by washing of each mouse peritoneum with 5 ml of medium 199. Suspension of PEC in the volume of 10 ml was placed in a 100 mm Petri dish and then incubated for 2 hours at 37° C. and 5% CO₂. Non-adherent cells were removed, while adherent cells were collected from the surface of the dish using a rubber policeman, and then the concentration of PEC was adjusted to 2mln cells in 50 μl volume. This volume of suspension was placed into well of 96-well microtitre plate, then 5′-adenosine monophosphate was added as a substrate of 5′-NTDase, and cells were incubated for 60 minutes under the above described conditions. The enzyme activity was measured using photometry (λ620 nm), according to the colouring intensity of a molybdenum reagent, which was placed in all wells following the incubation. The results were shown as units of optical density per 1 mln PEC and as relative 5′-NTDase activity in percent of control level. The activity of 5′-NTDase in the presence of APG was 16% of the control level.

### EXAMPLE 10

Activation of Human Immune System

This Example shows the in vitro influence of APG on immune system cells, circulating in human peripheral blood.

Activation of Human NK-cells

Appearance of the activation marker as a result of APG influence on human NK-cells was found using three-colour laser cytometry. Heparinised blood was taken by vein puncture of healthy donors using the ‘Vacutainer’ system (produced by Becton Dickinson). APG preparation was diluted to a concentration of 1 mg/ml in medium RPMI-1640 and was sterilised by filtration through 0.22 mcm filter. Factor 2 serial concentrations of 0.016 to 100 μg/ml APG were prepared in medium RPMI-1 640 and placed as a volume of 0.2 ml in wells of 48-well Nunc culture plate. As a negative control, 0.2 ml of RPMI-1640 without APG was used. 0.2 ml of heparinised blood was added into each well. The samples were incubated at 37° C. and 5% CO₂ for 3-48 hours. After incubation, 0.05 ml of the blood sample was placed into a 1.2 ml plastic tube. 5 μl of the following antibodies were inserted: anti-CD16-FITC (Sorbent), anti-CD69-phycoerithrine (Pharmingen), anti-CD3-PerCR (Becton Dickinson). The tubes were shaken for 5 seconds, and then were incubated at room temperature in the dark over 20 minutes. 1 ml of lysing-fixating solution (Becton Dickinson) was added into each of the test tubes, which were then kept for 15 minutes at room temperature. The test tubes were centrifuged for 10 min at 300 g, the supernatant liquid was removed, and the cell pellet was re-suspended in 0.5 ml of isotonic solution.

Use of a laser-activated fluorescence cytofluorimeter (FACS Calibur, produced by Becton Dickinson), and using Cell Quest software, showed that only 7% of NK-cells expressed CD69, an activation marker, in the absence of APG, but practically all (96%) of the NK-cells were activated and expressed CD69, if 10 μg/ml APG was present in the cell cultures.

Increase in Cytolytic Activity of NK-cells Under Influence of APG

A mononuclear fraction was obtained from heparinised blood using Ficoll (Pharmacia) density gradient centrifugation (30 minutes, 300 g at room temperature). Cells were washed with medium 199 and suspended at 2 mln/ml in the complete medium (CM) comprising RPMI-1640 supplemented with 10% foetal bovine serum, 20 mM Hepes buffer (pH 7.4) and 10 μg/ml of gentamycine. 1 ml of cell suspension was placed into each well of a 12-well Nunc culture plate, and then 1 ml of CM (control), 1 ml of APG solution at 20 μg/ml, or 1 ml of interleukin-2 (IL-2,20 ME/ml, a standard NK-cell activator) was added in the cell cultures. The cultures were incubated for 3 hours at 37° C. in an atmosphere of 5% CO₂. After incubation, the cells were collected in the centrifuge tubes, settled by centrifugation (10 minutes, 300 g), and re-suspended at a concentration of 5 min cells per 1 ml CM. The recovered cells served as cytolytic effectors. Triplets of serial 2-fold dilutions of effector cells in CM were introduced (volume 0.1 ml) into wells of a 96-well round-bottomed culture plate. 10,000 of ³ H-uridine-labelled target cells of K562 cell line origin were added in each culture well. The mixture of effector and target cells was incubated for 4 hours at 37° C. in the atmosphere of 5% CO₂. After incubation, the cells were transferred onto paper filters using Titertek Cell Harvester 550, and then radioactivity levels were counted using a Wallac 1409 meter.

Following 3 hrs incubation of the effector cells in the presence of APG, 29% of NK-cells were expressing CD69, which meant cells were activated. A study of cytolytic activity of such APG-activated effector cells against K562 tumour target cells showed. a strong increase, under influence of APG, of the ability of NK-cells to kill target cells. This was more obvious at a low effector-to-target ratio, in particular at ratios 6.25 and 12.5, when the cytolytic capacity of NK cells increased three times under influence of APG. APG was more effective in activating of NK cells than IL-2, regardless of whether the activation was assessed according to the expression of CD69 or the killing activity against K-562 tumour cells.

Activation of Granulocytes

Study of an expression of activation markers on different types of cells showed that APG induced CD69 expression on granulocytes. Activation of granulocytes was obvious after 24 hours of in vitro incubation in the presence of APG. At this time 60% of granulocytes were expressing CD69.

### EXAMPLE 11

Activation of Synthesis and Secretion of Cytokines

This Example shows that APG activates cytokine production in human blood cells. For instance, APG initiates production of interleukin-1 beta (IL-1β, tumour necrosis factor alpha (TNF-α) and interleukin-8 (IL-8).

Heparinised blood was obtained by vein puncture of healthy donors using the Vacutainer system (Becton Dickinson). APG preparation was diluted to 1 mg/ml in RPMl-1640 and was sterilised by filtration through a 0.22 μm filter. Factor 2 serial concentrations of 0.016 to 100 μg/ml APG were prepared in RPMl-1640 and placed at a volume of 0.2 ml in wells of a Nunc 48-well culture plate. As a negative control, 0.2 ml of RPMl-1640 without APG was used. Then 0.2 ml of heparinised blood was added into each well.

The samples were incubated at 37° C. and 5% CO₂ for 3-48 hours. After the incubation, 0.2 ml of culture medium was collected in 0.5 ml centrifuge test tubes, and was centrifuged at 10000 rpm for 15 minutes. Supernatant was frozen at −70° C. and used to determine the secretory cytokines. The cells that remained in the culture well were gently pipetted and transferred into 1.2 ml plastic test tubes to determine the cell activation markers using flow cytometry. IL-1β concentration in the culture supernatant was measured using EIA. IL-1β kit produced by Immunotech (France) was used in accordance with manufacturer's instruction. TNF-α and IL-8 were measured using respective EIA kits from Innogenetics (Belgium). The colour intensities were measured using a automatic photometer (450 nm) reader manufactured by Dynatech (Switzerland).

Intracellular cytokines were detected in human blood monocytes using three-colour laser-activating flow cytometry (FACS Calibur, Becton Dickinson, USA). Heparinised blood was diluted 1:1 with RPMI-1640, containing various concentrations of APG and also 10 μg/ml brefeldin A (Sigma, USA), an inhibitor of protein secretion in eukaryotic cells. Samples were incubated for 5 hours at 37° C. and at 5% CO₂. After incubation, 0.1 ml of suspension was taken, and 1 ml of lysing-fixating solution (Becton Dickinson) was added. The mixture was kept for 15 minutes at room temperature. The test tubes were centrifuged (300g, 10 minutes), the supernatant was discarded and the cell pellet was suspended in 0.5 ml of cell permeabilization cocktail (Becton Dickinson). The suspension was kept for 10 minutes at room temperature. This procedure permits colouring. of the insides of cells. After adding 5 ml of isotonic liquid containing 0.5% of bovine serum and 0.1% of sodium azide, the cells were settled by centrifuge (300 g, 10 min), and the supernatant was discarded. 5 μl of antibodies were added to the cell pellet. For determining IL-1β within monocytes, the following mixture of antibodies was used: anti-IL-1β-FITC (Caltag Lab.), anti-CD14-PE (Caltag Lab.), and anti-CD45-PerCP (Becton Dickinson). Intracellular determination was similar, but using FITC-labelled anti-TNF-α in the respective mixture of labelled antibodies.

Results showed that APG stimulates production of TNF-α, IL-1β and IL8 by monocytes, and that monocytes are a source of these cytokines in extracellular medium. Indeed, if brefeldin A was added into the incubation medium and hence secretion of proteins was blocked, and accumulation of these cytokines inside monocytes was recorded using flow cytometry, their concentration in the culture medium was at background level. Without brefeldin A, the accumulation of the cytokines in monocytes was not obvious since they were constantly exported to outside the cell, and hence the concentration of the cytokines in the culture medium dramatically increased, being in direct dependence on the concentration of APG. Dose-dependence curves showed an exponential growth of TNF-α, IL-1β and IL-8 production by monocytes with the increase of APG-concentration in vitro from 40 to 400 ng/ml.

### EXAMPLE 12

Induction of Interferon Synthesis

A study of interferon-inducing activity of APG was performed in vitro using human cells lines, namely L41, a fibroblastoid, and J-96, a monocytoid cell lines. The intensity of interferon induction was assessed by the anti-viral activity of APG on human cell lines infected with encephalo-myocarditis virus (EMV). 20,000 cells per 100 μl volume were placed into each well of a 96-well flat-bottomed culture plate. Medium 199 was used for cultures of J-96 cells, while L41 were grown using Eagle's minimum essential medium; both media were supplemented with 10% foetal bovine serum, 300 μg/ml L-glutamine and 100 unit/ml penicillin.

Ridostin (Vector, Russia), a double-stranded microbial DNA preparation, was used as a reference interferon-inducer. Interferon-inducing efficacy of APG and Ridostin was assessed according to their ability to in vitro protect L-41 and J-96 cells from the cytopathogenic influence of EMV. Interferon-inducing capacity was expressed as a minimum effective concentration (CPD₅₀) of the compound protecting 50% of cells from the cell death caused by the virus.

Factor 2 serial dilutions of the preparation tested were prepared in the cell cultures to a final concentration of APG or Ridostin from 0 to 100 μg/ml. Viability of the infected cells and cytopathogenic effect of the virus were counted using a light microscope (Leitz, magnification 200×) after 24 hours of infection. Results are shown in Tables 3 and 4, for 3 experiments in each case.

TABLE 3
Interferon-inducing efficacy of APG in L-41 cells cultures
infected with 100LD₁₀₀ EMV

| | ANTI-VIRAL EFFECT (CPD₅₀) | | | |
|---|---|---|---|---|
| PREPARATIONS | Experiment 1 | Experiment 2 | Experiment 3 | M +− m |
| APG | 100 μg/ml | 50 μg/ml | 50 μg/ml | 66.7 ± 16.7 μg/ml |
| Ridostatin | 250 μg/ml | 4 μg/ml | 15.6 μg/ml | 89.9 ± 80.1 μg/ml |

TABLE 4
Interferon-inducing efficacy of APG in J-96 cells cultures
infected with 100LD₁₀₀ EMV

| | ANTI-VIRAL EFFECT (CPD₅₀) | | | |
|---|---|---|---|---|
| PREPARATIONS | Experiment 1 | Experiment 2 | Experiment 3 | M +− m |
| APG | 25 μg/ml | 12.5 μg/ml | 6.25 μg/ml | 14.6 ± 5.5 μg/ml |
| Ridostatin | 2 μg/ml | 8 μg/ml | 8 μg/ml | 6 ± 2 μg/ml |

### EXAMPLE 13

Activation of Antibacterial Defence

APG was tested as an enhancer of anti-bacterial defence using Salmonella typhimurium experimental infection in mice. The experiments were performed on mice of both sexes, weighing 12-14g. Different doses (3.3 μg, 10 μg or 30 μg) of APG were injected subcutaneously 24 hours prior to infection. Mice were challenged intraperitoneally with 10², 10³, 10⁴ or 10⁵ microbe cells (per mouse). Mortality was recorded over 20 days. Protective efficacy of the preparation was assessed according to increase of animals' survival (%), the infection dose (ID₅₀) causing 50% death, and prolongation of the life span after the challenge. Results are shown in Table 5.

TABLE 5
Influence of APG on the resistance of mice
to infection with Salmonella typhimurium

| | | | | | Average |
|---|---|---|---|---|---|
| | | | | | life |
| | | Route of | Survival | | span |
| Preparation | Dose | injection | (%) | ID₅₀ | (days) |
| Physiological | 0.5 ml | S/c | 30 | 501 | 11.4 |
| saline | | | | (200-1258)* | |
| APG | 3.3 μg | S/c | 32.5 | 630 | 12 |
| | | | | (251-2512) | |
| | 10 μg | S/c | 37.5 | 1000 | 11.6 |
| | | | | (316-3162) | |
| | 30 μg | S/c | 52.5 | 3981 | 13.1 |
| | | | | (1258-12589) | |

*mean ID₅₀ and its limits (in brackets) with probability of 0.05 are indicated.

It has also been found that APG successfully increases the anti-infective defence against staphylococcus in chicken. Further, immunomodulation using APG can increase the efficacy of anti-bacterial chemotherapy. Nifulin, an anti-bacterial preparation, was introduced to chickens at a dose of 10 mg/kg per os, 20 minutes after challenge with 1.5×10⁹ Staphylococcus aureus. The therapy using nifulin increased the survival rate of chickens up to 70%, in comparison to 40% in the challenge control group. The combination of APG and nifulin defended 100% of the infected chickens.

### Example 14

Activation of Anti-viral Defence

Anti-viral effect of APG was studied in white non-inbred mice using an experimental model of Herpes simplex virus type 1 (HSV-1) infection. Results showed preventative and therapeutic effects of APG. An injection of 3 μg APG per mouse 48 hours before challenge or 3-30 μg per mouse, twice, 48 and 24 hours before challenge, protected 40-60% of animals from death because of the infection.

The capability of APG to increase human cells' resistance to the viral infection was studied using the HSV-1 infection in VERO cell cultures. It was shown that APG conferred on the VERO cells protection from cell death caused by HSV-1.

The virus replication was dramatically suppressed within the VERO cells treated using APG as compared to untreated VERO cells. Thus, the HSV-1 titre was reduced by a factor of 100-1000, if the VERO cells were pre-treated using APG 24 hours before the challenge. If the preparation was administered after the challenge of VERO cells with the HSV-1, the titre of the virus was reduced by 70-100 times.

1. A substance having immunostimulating, anti-viral and/or anti-bacterial activities, obtainable by extraction from mashed plant raw material of the Dioscoreaceae, Plantaginaceae or Solanaceae family with water, and which is a water-soluble acidic peptidoglycan having a molecular weight of 1200-40000 kD, and a glucose to uronic acid weight ratio of 1 to 2-4.

2. The substance according to claim 1, of which the peptide portion constitutes 13±3% of the total mass of acidic peptidoglycan.

3. The substance according to claim 1, which contains 10-15% of palmitic and stearic acids in a ratio of 2.4:1.

4. (cancel).

5. A pharmaceutical composition comprising a substance having immunostimulating, anti-viral and/or anti-bacterial activities, obtainable by extraction from mashed plant raw material of the Dioscoreaceae, Plantaginaceae or Solanaceae family with water, and which is a water-soluble acidic peptidoglycan having a molecular weight of 1200-40000 kD, and a glucose to uronic acid weight ratio of 1 to 2-4, and a pharmaceutically acceptable carrier.

6. The composition according to claim 5, in liquid form.

7.-13. (cancel).

14. A therapeutic method for treating a patient wherein said method comprises administering to the patient a substance having immunostimulating, anti-viral and/or anti-bacterial activities, obtainable by extraction from mashed plant raw material of the Dioscoreaceae, Plantaginaceae or Solanaceae family with water, and which is a water-soluble acidic peptidoglycan having a molecular weight of 1200-40000 kD, and a glucose to uronic acid weight ratio of 1 to 2-4.

15. The therapeutic method, according to claim 14, for treating a condition selected from the group consisting of:
a) conditions that can be ameliorated by enhanced antibody synthesis;
b) conditions that can be ameliorated by activated macrophages;
c) conditions that can be ameliorated by activated monocytes;
d) conditions that can be ameliorated by activated natural killer cells (NK-cells);
e) conditions that can be ameliorated by activated granulocytes;
f) conditions that can be ameliorated by activated cytokine synthesis; and
g) conditions that can be ameliorated by activated interferon synthesis.

16. The method, according to claim 15, for treating a condition that can be by enhanced antibody synthesis.

17. The method, according to claim 15, for treating a condition that can be ameliorated by activated macrophages.

18. The method, according to claim 15, for treating a condition that can be by activated monocytes.

19. The method, according to claim 15, for treating a condition that can be by activated natural killer cells (NK-cells).

20. The method, according to claim 15, for treating a condition that can be by activated granulocytes.

21. The method, according to claim 15, for treating a condition that can be by activated cytokine synthesis.

22. The method, according to claim 15, for treating a condition that can be by activated interferon synthesis.

23. the method according to claim 14, wherein the peptide portion of said substance constitute 13±3% of the total mass of acidic peptidoglycan.

24. The method, according to claim 14, wherein said substance contains 10-15% of palmitic and stearic acids in a ratio of 2.4:1.
